SEQUENCE LISTING (27 sequences)

SEQ ID NO: 1 - protein, 7 aa, Artificial Sequence
  MOD_RES (1) . . (7): The peptide is labelled with a radionuclide or with a paramagnetic metal isotope.
YMGWMDX

SEQ ID NO: 2 - protein, 8 aa, Artificial Sequence
  MOD_RES (1) . . (8): The peptide is labelled with a radionuclide or with a paramagnetic metal isotope.
DYMGWMDX

SEQ ID NO: 3 - protein, 8 aa, Artificial Sequence
  MOD_RES (1) . . (8): The peptide is labelled with a radionuclide or with a paramagnetic metal isotope.
DYXGWXDX

SEQ ID NO: 4 - protein, 8 aa, Artificial Sequence
  MOD_RES (1) . . (8): The peptide is labelled with a radionuclide or with a paramagnetic metal isotope.
XYXGWXDX

SEQ ID NO: 5 - protein, 8 aa, Artificial Sequence
  MOD_RES (1) . . (8): The peptide is labelled with a radionuclide or with a paramagnetic metal isotope.
XYMGWMDX

SEQ ID NO: 6 - protein, 8 aa, Artificial Sequence
  MOD_RES (1) . . (8): The peptide is labelled with a radionuclide or with a paramagnetic metal isotope.
XYXGWXDX

SEQ ID NO: 7 - protein, 8 aa, Artificial Sequence
  MOD_RES (l) . . (8): The peptide is labelled with a radionuclide or with a paramagnetic metal isotope.
DYTGWXDX

SEQ ID NO: 8 - protein, 9 aa, Artificial Sequence
  MOD_RES (1) . . (9): The peptide is labelled with a radionuclide or with a paramagnetic metal isotope.
RDYXGWXDX

SEQ ID NO: 9 - protein, 9 aa, Artificial Sequence
  MOD_RES (1) . . (9): The peptide is labelled with a radionuclide or with a paramagnetic metal isotope.
RDYTGWXDX

SEQ ID NO: 10 - protein, 10 aa, Artificial Sequence
  MOD_RES (1) . . (10): The peptide is labelled with a radionuclide or with a paramagnetic metal isotope.
YGDYXGWXDX

SEQ ID NO: 11 - protein, 10 aa, Artificial Sequence
  MOD_RES (1) . . (10): The peptide is labelled with a radionuclide or with a paramagnetic metal isotope.
XGDYXGWXDX

SEQ ID NO: 12 - protein, 10 aa, Artificial Sequence
  MOD_RES (1): Xaa is DTyr.
XGDYXGWXDX

SEQ ID NO: 13 - protein, 10 aa, Artificial Sequence
  MOD_RES (1): Xaa is 125I iodinated DTyr.
XGDYXGWXDX

SEQ ID NO: 14 - protein, 10 aa, Artificial Sequence
  MOD_RES (1): Xaa is DTyr.
XGDXXGWXDX

SEQ ID NO: 15 - protein, 10 aa, Artificial Sequence
  MOD_RES (1): Xaa is 125I iodinated DTyr.
XGDXXGWXDX

SEQ ID NO: 16 - protein, 8 aa, Artificial Sequence
  MOD_RES (2): Xaa is Tyr(SO3H).
DXMGWMDX

SEQ ID NO: 17 - protein, 8 aa, Artificial Sequence
  MOD_RES (8): Xaa is Phe-NH2.
DYMGWMDX

SEQ ID NO: 18 - protein, 10 aa, Artificial Sequence
  MOD_RES (1): Xaa is DTyr.
XGDXXGWXDX

SEQ ID NO: 19 - protein, 8 aa, Artificial Sequence
  MOD_RES (1): Xaa is DTPA substituted Asp.
XYMGWMDX

SEQ ID NO: 20 - protein, 8 aa, Artificial Sequence
  MOD_RES (1): Xaa is DTPA substituted Asp.
XYXGWXDX

SEQ ID NO: 21 - protein, 8 aa, Artificial Sequence
  MOD_RES (1): Xaa is DTPA substituted DAsp.
XYXGWXDX

SEQ ID NO: 22 - protein, 8 aa, Artificial Sequence
  MOD_RES (1): Xaa is DTPA substituted DAsp.
XYMGWMDX

SEQ ID NO: 23 - protein, 8 aa, Artificial Sequence
  MOD_RES (1): Xaa is beta-DTPA substituted Dpr.
XYXGWXDX

SEQ ID NO: 24 - protein, 8 aa, Artificial Sequence
  MOD_RES (1): Xaa is DTPA substituted Asp.
XYTGWXDX

SEQ ID NO: 25 - protein, 8 aa, Artificial Sequence
  MOD_RES (1): Xaa is ll5Indium-DTPA substituted Asp.
XYXGWXDX

SEQ ID NO: 26 - protein, 8 aa, Artificial Sequence
  MOD_RES (1): Xaa is ll5Indium-DTPA substituted DAsp.
XYXGWXDX

SEQ ID NO: 27 - protein, 33 aa, Artificial Sequence
  SITE (1) . . (25): The first 25 Xaa′s may or may not be present; can be equal or different; are selected from Ala, Leu, Asn, Dpr, Gln, Glu, Ser, Ile, Met, His, Asp, Lys, Gly, Thr, Pro, Pyr, Arg, Tyr, Trp, Val and Phe.
XXXXXXXXXXXXXXXXXXXXXXXXXXYXGWXDX

(19) United States
(12) Patent Application Publication
(10) Pub. No.: US 2004/0185510 A1
(43) Pub. Date: Sep. 23, 2004

(54) Use of labelled CCK-B receptor ligands for the detection and localization of malignant human tumours

(76) Inventor: Jean-Claude Reubi, Wabern (CH)

Correspondence Address: BLACKWELL SANDERS PEPER MARTIN LLP, 720 OLIVE STREET, SUITE 2400, ST. LOUIS, MO, 63101, US

(21) Appl. No.: 10/626,229

(22) Filed: Jul. 24, 2003

Related U.S. Application Data
(63) Continuation of application No. 09/125,823, filed on Jan. 19, 1999, now abandoned.

(30) Foreign Application Priority Data
Feb. 27, 1996 (EP) 96200498.2
Feb. 25, 1997 (WO) PCT/US97/03056

(51) Int. Cl. A61K 51/00; G01N 33/574

(52) U.S. Cl. 435/7.23; 424/1.69

(57) ABSTRACT

The invention relates to a method of detecting and localizing malignant tumours and their metastases in tissues, which in healthy condition do not contain disturbing quantities of CCK-receptors, in the body of a human being, which comprises (i) administering to said being a composition comprising, in a quantity sufficient for external imaging, a labelled peptide derived from a compound of the general formula

$$( I )$$

$$H - {( Xaa )}_{n} - {( Xbb )}_{m} - Tyr - Xcc - {Gly}_{5} - Trp - Xdd - Asp - Phe - {R}_{2}$$

or an acid amide thereof, formed between a free NH₂-group of an amino acid moiety and R₁COOH, wherein

R₁ is a (C₁-C₃)alkanoyl group, an arylcarbonyl group, or an aryl-(C₁-C₃) alkanoyl group;

or a lactam thereof, formed between a free NH₂ group of an amino acid moiety and a free CO₂H group of another amino acid moiety; or a conjugate thereof with avidin or biotin;

wherein:

(Xaa)ₙ stands for 0 to 25 amino acid moieties which are equal or different and are selected from Ala, Leu, Asn, Dpr, Gln, Glu, Ser, Ile, Met, His, Asp, Lys, Gly, Thr, Pro, Pyr, Arg, Tyr, Trp, Val and Phe;

m=0 or 1;

Xbb is Asp, Dpr, Glu or Pyr, with the proviso that Xbb can only be Pyr when n=0;

Xcc is Met, Leu or Nle;

Xdd is Met, Leu or Nle; and

R₂ is a hydroxy group, an acetoxy group or an amino group; and thereupon (ii) subjecting said being to external imaging, by radioactive scanning or by magnetic resonance imaging, to determine the targeted sites in the body of said being. The invention further relates to a method for the therapeutic treatment of said malignant tumours by administration of the above-defined peptide, labelled for this purpose.

The invention also relates to a method for labelling of the peptide compounds, to a pharmaceutical composition to be used for detection, to a pharmaceutical composition to be used for therapy and to a kit for preparing a radiopharmaceutical composition.

The invention relates to a method of detecting and localizing malignant tumours in the body of a human being. The invention further relates to the therapeutic treatment of these tumours in the body of said being. The invention also relates to a pharmaceutical composition, to a labelled peptide to be used in this composition, and to a kit for preparing a pharmaceutical composition.

Cholecystokinin (CCK) is a neuropeptide that exerts numerous effects in the gastrointestinal tract and in the brain and is already known since a number of years. CCK is a member of a peptide family including also gastrin. CCK has been studied by several groups, mainly on its normal function in warm-blooded animals and humans. Povoski et al. (Oncology Research 1994, 6, 411-7) have studied the expression pattern of CCK receptors in the normal pancreas and in a pancreatic carcinoma in the rat and the mouse with the aid of the ¹²⁵I Bolton-Hunter labelled octapeptide CCK-8. Although it appears from this study that pancreatic carcinomas express CCK-receptors, this property cannot be used for the detection and localization of these carcinomas, as the normal tissue also expresses disturbing quantities of CCK receptors (see Tang et all., Gasteroenterology 1996, 111, 1621-28).

It is the objective of the present invention to provide for a method of detecting and localizing malignant tumours and their metastases in the body of a human being, in particular some specific tumours that are difficult to characterize. Examples of such malignant human tumours are Small Cell Lung Carcinoma (SCLC) and Medullary Thyroid Carcinoma (MTC).

Such a method would be a powerful tool, not only in diagnosing such tumours but also in supporting an effective therapy therefor. As a matter of fact, in order to be able to achieve a specific therapy for the control of such tumours, the detection and localization of these tumours, and in particular of the metastases thereof, in an early stage of their development is of utmost importance various requirements have to be imposed on an agent that is used in such a diagnostic method, such as non-toxic, no adverse influence on the host resistance and/or on the therapeutic treatment, well detectable and highly selective. The required high selectivity means that the diagnostic agent, after having been introduced into the body, must accumulate more strongly in the target tumours to be detected or visualized than in surrounding tissues. This selectivity, i.e. a comparatively stronger concentration of the diagnostic agent in the target tumours compared with non-target tissues, enables the user to correctly diagnose the malignancy. In order to be detectable from outside the body, the diagnostic agent should be labelled, preferably with a radionuclide or with a paramagnetic metal atom. In the former case, the radioactive radiation can be detected by using a suitable detector (scanning). Modern techniques in this field use emission tomography; when gamma radiating isotopes are used, the so-called single photon emission computerized tomography (SPECT) may be applied. The use of paramagnetic diagnostic agents enables a detection by means of imaging by magnetic resonance.

The above-defined objective can be achieved, according to the present invention, by a method of detecting and localizing malignant tumours and their metastases in tissues, which in healthy condition do not contain disturbing quantities of CCK-receptors, in the body of a human being, which comprises (i) administering to said being a composition comprising, in a quantity sufficient for external imaging, a peptide derived from a compound of the general formula

$$( I )$$

$$H - {( Xaa )}_{n} - {( Xbb )}_{m} - Tyr - Xcc - {Gly}_{5} - Trp - Xdd - Asp - Phe - {R}_{2}$$

or an acid amide thereof, formed between a free NH₂-group of an amino acid moiety and R₁COOH, wherein

R₁ is a (C₁-C₃)alkanoyl group, an arylcarbonyl group, or an aryl-(C₁-C₃)alkanoyl group; or a lactam thereof, formed between a free NH₂ group of an amino acid moiety and a free CO₂H group of another amino acid moiety; or a conjugate thereof with avidin or biotin;

wherein:

(Xaa)ₙ stands for 0 to 25 amino acid moieties which are equal or different and are selected from Ala, Leu, Asn, Dpr, Gln, Glu, Ser, Ile, Met, His, Asp, Lys, Gly, Thr, Pro, Pyr, Arg, Tyr, Trp, Val and Phe;

m=0 or 1;

Xbb is Asp, Dpr, Glu or Pyr, with the proviso that Xbb can only be Pyr when n=0;

Xcc is Met, Leu or Nle;

Xdd is Met, Leu or Nle; and

R₂ is a hydroxy group, an acetoxy group or an amino group;

said peptide being labelled with (a) a radioactive metal isotope selected from the group consisting of ^{99m}Tc, ²⁰³Pb, ⁶⁷Ga, ⁶⁸Ga, ⁷²As, ¹¹¹In, ^{113m}In, ⁹⁷Ru, ⁶²Cu, ⁶⁴Cu, ⁵² Fe, ^{52m}Mn and ⁵¹Cr, or (b) with a paramagnetic metal atom selected from the group consisting of Cr, Mn, Fe, Co, Ni, Cu, Pr, Nd, Sm, Yb, Gd, Tb, Dy, Ho and Er, or (c) with a radioactive halogen isotope, selected from ¹²³I, ¹³¹I, ⁷⁵Br, ⁷⁶Br, ⁷⁷Br and ⁶²Br, and thereupon (ii) subjecting said being to external imaging, by radioactive scanning or by magnetic resonance imaging, to determine the targeted sites in the body of said being in relation to the background activity, in order to allow detection and localization of said tumours in the body.

It has been found that certain carcinomas and sarcomas in tissues of a human being outside the gastrointestinal tract and the brain, that normally are not expressing CCK-receptors, do contain detectable amounts of CCK receptors. Examples of these tumours are Small Cell Lung Carcinoma (SCLC)(Denyer et al, Eur. J. of Pharm. 1994, 268, 29-41), Medullary Thyroid Carcinoma (MTC), Breast Carcinoma, Stromal Ovarian Carcinoma, Muscle Sarcoma. It has surprisingly been found that the CCK analogs will preferentially recognize CCK-A or CCK-B receptor-expressing tumours depending on the sulfation state: Unsulfated CCK and analogs will specifically recognize CCK-B receptors, and are therefore, after labelling, suitable compounds for the detection of tissues having CCK-B receptors. The normal sulfated CCK and analogs are recognizing both CCK-A and CCK-B receptors.

The present invention understands by CCK receptors CCK-B receptors that are found preferentially in the above mentioned tumours, whereas they are rarely expressed in Non-Small Cell Lung Carcinoma (NSCLC) and Non-Medullary Thyroid Cancers. CCK-A receptors are usually not or rarely expressed by first mentioned tumours that are expressing CCK-B receptors. According to the present invention the CCK-B receptors can be specifically labelled with adequate CCK analogs, as described below.

The above labelled peptides have been tested in suitable model experiments that are predictive for in vivo application. In these model experiments human tumourgti samples are used to mimic in vivo application. The experiments are described in the Examples appended. From the results it will be evident that the tested labelled peptides, even after drastic changes caused by attaching of a (metal containing) chelating group, have properties which make them pre-eminently suitable for the specific detection and localization of the above mentioned malignant human tumours expressing CCK-B receptors, especially for the specific detection of SCLC and MTC, even in the presence of tissue containing CCK-A receptors.

Suitable examples of aryl groups in R₁ are: phenyl, substituted phenyl or indolyl; preferably phenyl, 4-fluorophenyl, 2- or 4-bromo-phenyl, 2-iodophenyl, 4-hydroxyphenyl, 3-iodo-4-hydroxyphenyl, 4-fluoro-2-bromophenyl and 4-fluoro-2-iodophenyl.

In the case of the use of a conjugate of the peptide with avidin or biotin, the label is attached subsequently by reaction with labelled biotin in the case of avidin-conjugated peptide as described by Kalofonos et al. (J. Nucl. Med. 1990, 31, 1791), or by reaction with labelled avidin in the case of biotin-conjugated peptide as described by Paganelli et al. (Int. J. Cancer 1988, 2, 121).

In the above labelled peptide compounds one or more of the amino acids may have the D-configuration instead of the normal L-configuration.

The labelled peptide compounds of the invention may also comprise so-called pseudo peptide bonds, viz. —CH₂—NH— bonds, in addition to the natural amide bonds, viz. —CO—NH— bonds. Such modifications of the amino acids naturally occurring in peptides are within the scope of the present invention.

It is another objective of the present invention to provide a method of intraoperatively detecting and localizing malignant tumours in tissues, which in healthy condition do not contain disturbing quantities of CCK-receptors, in the body of a human being.

This objective can be achieved, according to a different aspect of the present invention by (i) administering to said being a composition comprising, in a quantity sufficient for detection by a gamma detecting probe, a peptide derived from a compound of the general formula I as defined above or an acid amide thereof, formed between a free NH₂-group of an amino acid moiety and R₁COOH;

or a lactam thereof, formed between a free NH₂ group of an amino acid moiety and a free CO₂H group of another amino acid moiety; or a conjugate thereof with avidin or biotin; wherein (Xaa)ₙ, Xbb, Xcc, Xdd, m, R₁ and R₂ have the meanings defined above, said peptide being labelled with ¹⁶¹Tb, ¹²³I or ¹²⁵I and thereupon (ii), after allowing the active substance to be bound and taken up in said tumours and after blood clearance of radioactivity, subjecting said being to a radioimmunodetection technique in the relevant area of the body of said being, by using a gamma detecting probe.

It is another objective of this invention to provide a method for the differential diagnosis of selected tumours. Certain tumours (i.e. SCLC or MTC) originating in a defined organ (lung resp. thyroid) could be preferentially identified due to their expression of CCK-B receptors, according to the present invention, in contrast to NSCLC or Non-Medullary Thyroid cancers. The present invention allows therefore a non-invasive diagnosis of lung or thyroid cancers in particular.

It is still another objective of the present invention to provide a method for the therapeutic treatment of malignant tumours in tissues, which in healthy condition do not contain substantial quantities of CCK-receptors, in the body of a human being. This objective can be achieved, according to a further aspect of the present invention, by administering to said being a composition comprising, in a quantity effective for combating or controlling tumours, a peptide derived from a compound of the general formula I as defined above or an acid amide thereof, formed between a free NH₂-group of an amino acid moiety and R₁COOH;

or a lactam thereof, formed between a free NH₂ group of an amino acid moiety and a free CO₂H group of another amino acid moiety; or a conjugate thereof with avidin or biotin; wherein (Xaa)ₙ, Xbb, Xcc, Xdd, m, R₁ and R₂ have the same meanings defined above, said peptide being labelled with an isotope selected from the group consisting of ¹⁸⁶Re, ¹⁸⁸Re, ⁷⁷As, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹ Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁵Yb, ¹⁷⁷Lu, ¹⁰⁵Rh, ¹¹¹Ag, 124I and ¹³¹I.

Suitable examples of the above-defined peptides, which after labelling can be used in the method of the invention, are unsulfated CCK₇ and the corresponding CCK₈, CCK₉ and CCK₁₀-analogs. In formulas:

(1) unsulfated CCK₇ (=Tyr²⁷-CCK (27-33): H-Tyr-Met-Gly-Trp-Met-Asp-Phe-NH₂

(2) unsulfated CCK₈ (=Tyr²⁷-CCK (26-33): H-Asp-Tyr-Met-Gly-Trp-Met-Asp-Phe-NH₂

(3) unsulfated CCK₈-analog 1 (=Tyr ²⁷,Nle^{28,31}-CCK (26-33)): H-Asp-Tyr-Nle-Gly-Trp-Nle-Asp-Phe-NH₂

(4) unsulfated CCK₈-analog 2 (=DAsp²⁶,Tyr²⁷ Nle^{28,31}-CCK (26-33)): H-DAsp-Tyr-Nle-Gly-Trp-Nle-Asp-Phe-NH₂

(5) unsulfated CCK₈-analog 3 (=DAsp²⁶,Tyr²⁷-CCK (26-33)): H-DAsp-Tyr-Met-Gly-Trp-Met-Asp-Phe-NH₂

(6) unsulfated CCK₈-analog 4 (=Dpr²⁶,Tyr²⁷, Nle^{28,31}-CCK (26-33)): H-Dpr-Tyr-Nle-Gly-Trp-Nle-Asp-Phe-NH_{2 (}7) unsulfated CCK₈-analog 5 (=Tyr²⁷, Thr²⁸, Nle³¹-CCK (26-33)): H-Asp-Tyr-Thr-Gly-Trp-Nle-Asp-Phe-NH₂

(8) unsulfated CCK₉-analog 1 (=Tyr²⁷, Nle^{28,31}-CCK (25-33)): H-Arg-Asp-Tyr-Nle-Gly-Trp-Nle-Asp-Phe-NH₂

(9) unsulfated CCK₉-analog 2 (=Tyr²⁷, Thr²⁸, Nle³¹-CCK (25-33)): H-Arg-Asp-Tyr-Thr-Gly-Trp-Nle-Asp-Phe-NH₂

(10) unsulfated CCK₁₀-analog 1 (=Tyr²⁷, Nle^{28,31}-CCK (24-33)): H-Tyr-Gly-Asp-Tyr-Nle-Gly-Trp-Nle-Asp-Phe-NH₂

(11) unsulfated CCK₁₀-analog 2 (=DTyr²⁴, Tyr²⁷, Nle^{28,31}-CCK (24-33)): H-DTyr-Gly-Asp-Tyr-Nle-Gly-Trp-Nle-Asp-Phe-NH₂

If the peptide as defined above is labelled with a radioactive halogen atom, said radioactive halogen atom is preferably selected from the group consisting of ¹²³I, ¹²⁴I, ¹²⁵I, ¹³¹I, ⁷⁵Br, ⁷⁶Br, ⁷⁷Br and ⁸²Br, said radioactive halogen isotope being attached to a Tyr or Trp moiety of the peptide, or to the aryl group of substituent R₁.

If the peptide as defined above is labelled with a metal atom, said metal atom is preferably selected from (a) the group consisting of the radioactive isotopes ^{99m}Tc, ²⁰³Pb, ⁶⁷Ga, ⁶⁸Ga, ⁷²As, ¹¹¹ In, ^{113m}In, ⁹⁷Ru, ⁶²Cu, ⁶⁴Cu, ⁵²Fe, ^{52m}Mn, ⁵¹Cr, ¹⁸⁶Re, ¹⁸⁸Re, ⁷⁷As, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁶⁶Ho, ¹⁷²Tm, ¹⁶⁹Yb, ¹⁷⁵Yb, ¹⁷⁷Yb, ¹⁷⁷Lu, ¹⁰⁵Rh and ¹¹¹Ag; or (b) the group consisting of the paramagnetic metal ions Cr, Mn, Fe, Co, Ni, Cu, Pr, Nd, Sm, Yb, Gd, Tb, Dy, Ho, and Er; said metal atom being attached to the peptide by means of a chelating group chelating said atom, which chelating group is bound by an amide bond or through a spacing group to the peptide molecule.

Suitable chelating groups for chelating said metal atom are NₜS₍₄₋ₜ₎ tetradentate chelating agents, wherein t=2-4, or groups derived from ethylene diamine tetra-acetic acid (EDTA), diethylene triamine penta-acetic acid (DTPA), cyclohexyl 1,2-diamine tetra-acetic acid (CDTA), ethyleneglycol-0,0′-bis(2-aminoethyl)-N,N,N′,N′-tetra-acetic acid (EGTA), N,N-bis(hydroxybenzyl)-ethylenediamine-N,N′-diacetic acid (HBED), triethylene tetramine hexa-acetic acid (TTHA), 1,4,7,10-tetraazacyclododecane-N,N′,N″,N′″-tetra-acetic acid (DOTA), hydroxyethyldiamine triacetic acid (HEDTA), 1,4,8,11-tetra-azacyclo-tetradecane-N,N′,N″,N′″-tetra-acetic acid (TETA), substituted DTPA, substituted EDTA, or from a compound of the general formula

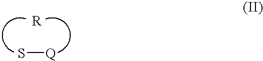

wherein R is a branched or non-branched, optionally substituted

hydrocarbyl radical, which may be interrupted by one or more hetero-atoms selected from N, O and S and/or by one or more NH groups, and

Q is a group which is capable of reacting with an amino group of the peptide and which is preferably selected from the group consisting of carbonyl, carbimidoyl, N-(C₁-C₆)alkylcarbimidoyl, N-hydroxycarbimidoyl and N-(C₁-C₆)alkoxycarbimidoyl.

NₜS₍₄₋ₜ₎ chelating agents, wherein t=2-4, are preferably selected from

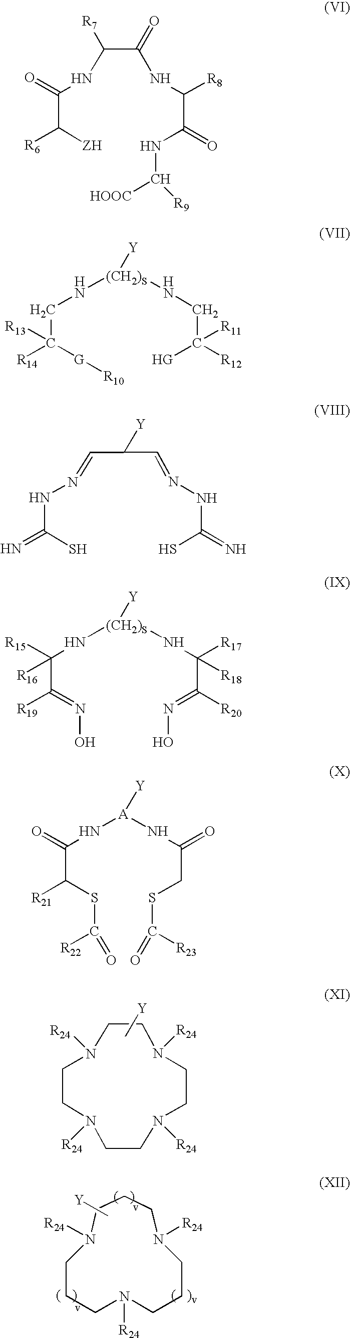

wherein:

R₆-R₂₀ are each individually hydrogen atoms or (C₁-C₄)alkyl groups, with the proviso that at least one of C₆ to C₉ is the symbol Y;

R₂₁ is a hydrogen atom or a CO₂(C₁-C₄)alkyl group;

R₂₂ and R₂₃ are each individually (C₁-C₄)alkyl groups or phenyl groups;

v is 0 or 1;

s is 2 or 3;

R₂₄ is CH₂COOH or a functional derivative thereof;

A is (C₁-C₄)alkylene, if desired substituted with CO₂alkyl, CH₂COalkyl, CONH₂, CONHCH₂CO₂alkyl; phenylene, phenylene substituted by CO₂alkyl, wherein the alkyl groups have 1 to 4 carbon atoms;

is G is NH or S;

Y is a functional group capable of binding with a free amino group of the peptide or with the spacing group;

and Z is S or O.

Said functional group Y preferably comprises isocyanato, isothiocyanato, formyl, o-halonitrophenyl, diazonium, epoxy, trichloro-s-triazinyl, ethyleneimino, chlorosulfonyl, alkoxycarb-imidoyl, (substituted or unsubstituted) alkylcarbonyloxycarbonyl, alkylcarbonylimidazolyl, succinimido-oxycarbonyl; said group being attached to a (C₁-C₁₀)hydrocarbon biradical.

Suitable examples of hydrocarbon biradicals are biradicals derived from benzene, (C₁-C₆)alkanes, (C₂-C₆)alkenes and (C₁-C₄)-alkylbenzenes.

Examples of suitable chelators of the general formula II are described in the international patent application WO 89/07456, such as unsubstituted or substituted 2-imino-thiolanes and 2-imino-thiacyclohexanes, in particular 2-imino-4-mercaptomethylthiolane.

Suitable examples of spacing groups, if present in the metal-labelled peptide molecule, are groups of the general formula

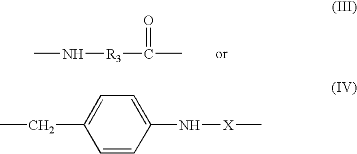

wherein R₃ is a C₁-C₁₀ alkylene group, a C₁-C₁₀ alkylidene group or a C₂-C₁₀ alkenylene group, and X is a thiocarbonyl group or a group of the general formula

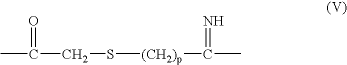

wherein p is 1-5.

Conjugates with avidin or biotin are formed as described by Paganelli et al. (Int. J. Cancer 1988, 2, 121), Kalofonos et al. (J. Nucl. Med. 1990, 31, 1791) and Anderson et al. (FEBS LETT. 1991, 282/1, 35-40).

The invention further relates to a pharmaceutical composition to be used for the above-defined method, comprising in addition to a pharmaceutically acceptable carrier material, preferably a physiological saline solution, and, if desired, at least one pharmaceutically acceptable adjuvant, as the active substance a peptide derived from a compound of the general formula I as defined above or an acid amide thereof, formed between a free NH₂-group of an amino acid moiety and R₁COOH;

or a lactam thereof, formed between a free NH₂ group of an amino acid moiety and a free CO₂H group of another amino acid moiety;

or a conjugate thereof with avidin or biotin; wherein (Xaa)₁, Xbb, Xcc, Xdd, m, R₁ and R₂ have the same meanings as defined above, said peptide being labelled with (a) a radioactive metal isotope selected from the group consisting of ^{99m}Tc, ²⁰³Pb, ⁶⁶Ga, ⁶⁷Ga, ⁶⁸Ga, ⁷²As, ¹¹¹In, ^{113m}In, ^{114m}In, ⁹⁷Ru, ⁶²Cu, ⁶⁴Cu, ⁵²Fe, ^{52m}Mn, ⁵¹Cr, ¹⁸⁶Re, ¹⁸⁸Re, ⁷⁷As, ⁹⁰Y, ⁶⁷ Cu, ¹⁶⁹Er, ^{117m}Sn, ¹²¹Sn, ¹²⁷Te, ¹⁴²Pr, ¹⁴³Pr, ¹⁹⁸Au, ¹⁹⁹Au, ¹⁴⁹Tb, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁶⁶Ho, ¹⁷²Tm, ¹⁶⁹Yb, Yb, ¹⁷⁵Lu, ¹⁰⁵Rh and ¹¹¹Ag, or (b) with a paramagnetic metal atom selected from the group consisting of Cr, Mn, Fe, Co, Ni, Cu, Pr, Nd, Sm, Yb, Gd, Tb, Dy, Ho and Er, or (c) with a radioactive halogen isotope, selected from ¹²³I, ¹³¹ I, ⁷⁵Br, ⁷⁶Br, ⁷⁷Br and ⁸²Br.

Suitable adjuvants are well-known in the art and include buffering agents such as HEPES buffer, TRIS buffer, etc., antioxidants and stabilizers such as ascorbic acid, gentisic acid or salts of these acids.

The invention also relates to a pharmaceutical composition to be used for the method of intraoperatively detecting and localizing malignant tumours as mentioned above, comprising in addition to a pharmaceutically acceptable carrier material and, if desired, at least one pharmaceutically acceptable adjuvant, as the active substance, in a quantity sufficient for intraoperatively detecting and localizing malignant tumours, a peptide selected from the group consisting of [¹²⁵I-D-Tyr]-Gly-Asp-Tyr-Nle-Gly-Trp-Nle-Asp-Phe-NH₂ and D-Tyr-Gly-Asp-[¹²⁵I-Tyr]-Nle-Gly-Trp-Nle-Asp-Phe-NH₂.

The invention also relates to the labelled peptide to be used as an active ingredient in the above pharmaceutical composition to be used in the above mentioned methods of detecting and localizing or therapeutic treatment of tumours and their metastases, said peptide being labelled with a metal atom as defined hereinbefore. Suitable chelating agents for chelating said metal atom are described above.

The invention also relates to the compounds [¹²⁵I-D-Tyr]-Gly-Asp-Tyr-Nle-Gly-Trp-Nle-Asp-Phe-NH₂ and D-Tyr-Gly-Asp-[¹²⁵]-Tyr-Nle-Gly-Trp-Nle-Asp-Phe-NH₂ especially to be used in the method of intraoperatively detecting and localizing malignant tumours

The invention also relates to a method of preparing a metal atom-labelled peptide as defined above, by reacting a derivatized peptide, comprising a peptide derived from a compound of the general formula I as defined in above or an acid amide thereof, formed between a free NH₂-group of an amino acid moiety and R₁COOH;

or a lactam thereof, formed between a free NH₂ group of an amino acid moiety and a free CO₂H group of another amino acid moiety; or a conjugate thereof with avidin or biotin; wherein (Xaa)ₙ, Xbb, Xcc, Xdd, m, R₁ and R₂ have the same meanings as defined above, derivatized with a chelating group bound by an amide bond or through a spacing group to the peptide molecule, with a metal atom as defined hereinbefore in the form of a salt or of a chelate, bound to a comparatively weak chelator, in order to form a complex.

The metal-labelled peptides of the invention can be prepared in a manner known per se for related compounds. For this purpose the peptide molecule is derivatized with the desired chelating agent as defined hereinbefore, e.g. NₜS_{[4-t]}, EDTA, DTPA, etc., directly or after introduction of a spacing group as defined above, after which the compound obtained is reacted with a metal isotope, as defined hereinbefore, in the form of a salt or of a chelate bound to a comparatively weak chelator, in order to form a complex.

Suitable examples of salts or chelates of the desired metal atom are: ¹¹¹In-oxinate, ^{99m}Tc-tartrate, etc. The complex-forming reaction can generally be carried out in a simple manner and under conditions that are not detrimental to the peptide.

The invention further relates to the results of the above preparation method, viz. a derivatized peptide, comprising a peptide derived from a compound of the general formula I as defined above or an acid amide thereof, formed between a free NH₂-group of an amino acid moiety and R₁COOH;

or a lactam thereof, formed between a free NH₂ group of an amino acid moiety and a free CO₂H group of another amino acid moiety;

or a conjugate thereof with avidin or biotin; wherein (Xaa)ₙ, Xbb, Xcc, Xdd, m, R₁ and R₂ have the same meanings as defined above, said peptide being derivatized with a chelating group bound by an amide bond or through a spacing group to the peptide molecule.

It is frequently impossible to put the ready-for-use composition at the disposal of the user, in connection with the often poor shelf life of the radiolabelled compound and/or the short half-life of the radionuclide used. In such cases the user will carry out the labelling reaction with the radionuclide in the clinical hospital or laboratory.

For this purpose the various reaction ingredients are then offered to the user in the form of a so-called “kit”. It will be obvious that the manipulations necessary to perform the desired reaction should be as simple as possible to enable the user to prepare from the kit the radioactive labelled composition by using the facilities that are at his disposal. Therefore the invention also relates to a kit for preparing a radiopharmaceutical composition.

Such a kit according to the present invention for preparing a radiopharmaceutical composition comprises (i) a derivatized peptide as defined above, to which derivatized peptide, if desired, an inert pharmaceutically acceptable carrier and/or formulating agents and/or adjuvants is/are added, (ii) a solution of a salt or chelate of a metal isotope selected from the group consisting of ²⁰³Pb, ⁶⁷Ga, ⁶⁸Ga, ⁷²As, ¹¹¹In, ^{113m}In, ⁹⁷ Ru, ⁶²Cu, ^{99M}Tc, ¹⁸⁶Re, ¹⁸⁸Re, ⁶⁴Cu, ⁵²Fe, ^{52m}Mn, ⁵¹Cr, ⁷⁷As, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁶⁶Ho, ¹⁷²Tm, ¹⁶⁹Yb, ¹⁷⁵Yb, ¹⁷⁷Lu, ¹⁰⁵Rh and ¹¹¹Ag, and (iii) instructions for use with a prescription for reacting the ingredients present in the kit.

Preferably the peptide compound to be used as an ingredient of the above kit has been derivatized by a reaction with a chelating agent as defined hereinbefore. The resulting peptide conjugate provides a facility for firmly attaching the radionuclide in a simple manner. Suitable chelating agents for modifying the peptide are described in detail hereinbefore. N-containing di- or polyacetic acids or their derivatives, such as the compounds mentioned before, have proved to be pre-eminently suitable for attaching various metal radionuclides, such as In-111 and In-113m, to the peptide molecules. The kit to be supplied to the user may also comprise the ingredient(s) defined sub (i) above, together with instructions for use, whereas the solution of a salt or chelate of the radionuclide, defined sub (ii) above, which solution has a limited shelf life, may be put to the disposal of the user separately.

In case the kit serves to prepare a radiopharmaceutical composition labelled with Tc-99m, Re-186 or Re-188, such a kit according to the present invention may comprise, in addition to the ingredient(s) defined sub (i) above, (ii) a reducing agent and, if desired, a chelator, and (iii) instructions for use with a prescription for reacting the ingredients of the kit with Tc-99m in the form of a pertechnetate solution, or with Re-186 or Re-188 in the form of a perrhenate solution. If desired, the ingredients of the kit may be combined, provided they are compatible. The kit should comprise a reducing agent to reduce the pertechnetate or perrhenate, for example, a dithionite, a metallic reducing agent or a complex-stabilizing reducing agent, e.g. SnCl₂, Sn(II)-tartrate, Sn(II)-phosphonate or -pyrophosphate, or Sn(II)-glucoheptonate. The pertechnetate or perrhenate solution can simply be obtained by the user from a suitable generator.

When the radionuclide is present in the kit itself, the complex forming reaction with the derivatized peptide can simply be produced by combining the components in a neutral medium and causing them to react. For that purpose the radionuclide may be presented to the derivatized peptide in the form of a chelate bound to a comparatively weak chelator, as described hereinbefore.

When the kit comprises a derivatized peptide as defined hereinbefore and is intended for the preparation of a radiopharmaceutical composition, labelled with Tc-99m, Re-186 or Re-188, the radionuclide will preferably be added separately in the form of a pertechnetate or perrhenate solution. In that case the kit will comprise a suitable reducing agent and, if desired, a chelator, the former to reduce the pertechnetate or the perrhenate. As a reducing agent may be used, for example, a dithionite or a metallic reducing agent. The ingredients may optionally be combined, provided they are compatible. Such a monocomponent kit, in which the combined ingredients are preferably lyophilized, is excellently suitable for being reacted, by the user, with the radionuclide solution. As a reducing agent for the above-mentioned kits is preferably used a metallic reducing agent, for example, Sn(II), Ce(III), Fe(II), Cu(I), Ti(III) or Sb(III); Sn(II) is excellently suitable. The peptide constituent of the above-mentioned kits, i.e. preferably the derivatized peptide, may be supplied as a solution, for example, in the form of a physiological saline solution, or in some buffer solution, but is preferably present in a dry condition, for example, in the lyophilized condition. When used as a component for an injection liquid it should be sterile, in which, when the constituent is in the dry state, the user should preferably use a sterile physiological saline solution as a solvent. If desired, the above-mentioned constituent may be stabilized in the conventional manner with suitable stabilizers, for example, ascorbic acid, gentisic acid or salts of these acids, or it may comprise other auxiliary agents, for example, fillers, such as glucose, lactose, mannitol, and the like.

The invention will now be described in greater detail with reference to the following specific Examples.

### EXAMPLE 1

### Preparation of Compound 12

The peptide DTyr-Gly-Asp-Tyr-Nle-Gly-Trp-Nle-Asp-Phe-NH₂ (Compound 12) is synthesised using the Chiron-Multipin Synthesis Technology (Geysen et al., Proc. Natl. Acad. Sci. 1964, 81, 3998-4002; Geysen et al., J. Immunol. Methods 1987, 102, 259-274). The peptide is analysed with HPLC (Merck Lichrosphere 100 RP-18, 250*4 mm, Gradient elution (A: 0.1% orthophosphoric acid in water; B: 0.1% orthophosphoric acid in 90% acetonitrile; 0-67% B in 15 minutes); Flow rate 1.5 ml; Detection wavelength 214 nm) and by Ion Spray Mass Spectrometry.

Results: purity (HPLC): 97.8%

Mw (IS-MS): 1247.3 (calculated 1247.4)

### EXAMPLE 2

### General Method for the Synthesis of CCK Analogs and DTPA containing CCK Analogs by Solid Phase Method and Synthesis of Compounds 19-24

Solid phase peptide synthesis (SPPS) is carried out using an Applied Biosystems Model 432 A Synergy Peptide synthesizer using Fmoc (9-fluorenemethoxycarbonyl) strategy. The general principles and methods followed are well known in the art. (see “Fluorenemethoxycarbonyl-polyamide solid phase synthesis-General Synthesis and Development”, Chapter 3 in “Solid Phase peptide synthesis—A practical approach” by E. Atherton and R. C. Sheppard, Information Press Ltd., Oxford, England (1989)). Three letter codes for common aminoacids are used. The unusal aminoacid 2,3-diaminopropionic acid has the abbreviation Dpr.

In the following examples, 9-fluorenemethoxycarbonyl (Fmoc)amino terminus protected amino acids are used. All the standard Fmoc-protected amino acids are purchased commercially unless stated. Coupling with dicyclohexyl-dicarbodiimide/hydroxybenzotriazole using Rink amide resin is used for carboxyl terminus amides. After the synthesis is completed, the products are routinely cleaved using a solution comprised of trifluoroacetic acid:phenol:thianisole:water (85:5:5:5) 6-10 hours at room temperature. The products are precipitated by t-butylmethylether and centrifuged. The mixture of solids containing the peptide and the resin is washed with t-butylmethylether and centrifuged five to six times to remove residual cleavage mixture (trifluoroacetic acid:phenol:thianisole:water (85:5:5:5)). Acetonitrile:water (2:3) mixture is added to the residue and filtered to remove the resin. Filtrate containing the crude peptide is lyophilized and pure peptides are obtained by preparative liquid chromatography. In this way the peptides corresponding to labelled compounds 1 to 11 can be prepared.

For the incorporation of DTPA (diehylenetriamine pentaacetic acid) the N-terminal Fmoc-protecting group is removed in the synthesizer using the standard protocol of the synthesizer and 3-4 molar equivalents tri-t-butyldiethylenetriaminepentacetic acid are used for the condensation to the N-terminal. Cleavage and deprotection are carried out as outlined above.

The following CCK derivatives were synthesized based on the above general procedure. The analyses were performed on a Finnigan TSQ-700 Triple Quad Mass Spectrometer with an Atmospheric Pressure Ionization interface. The samples were introduced by flow injection analysis into acetonitrile/water with 0.1 trifluoroacetic acid. Electrospray ionization was the mode of ionization employed and the instrument was run in positive ion mode.

1. DTPA-Tyr²⁷-CCK (26-33): DTPA-Asp-Tyr-Met-Gly-Trp-Met-Asp-Phe-NH₂ (Compound 19). Molecular Weight, Calculated: 1437.5, Found: 1438.8 (M⁺+1).

2. DTPA-Tyr²⁷,Nle^{28,31}-CCK(26-33): DTPA-Asp-Tyr-Nle-Gly-Trp-Nle-Asp-Phe-NH₂ (Compound 20). Molecular Weight, Calculated: 1401.6, Found: 1402.8 (M⁺+1).

3. DTPA-DAsp²⁶,Tyr²⁷, Nle^{28,31}-CCK(26-33): DTPA-DAsp-Tyr-Nle-Gly-Trp-Nle-Asp-Phe-NH₂ (Compound 21). Molecular Weight, Calculated: 1401.6, Found: 1402.8 (M⁺+1)

4. DTPA-DAsp²⁶, Tyr²⁷,-CCK(26-33): DTPA-DAsp-Tyr-Met-Gly-Trp-Met-Asp-Phe-NH₂ (Compound 22). Molecular Weight, Calculated: 1437.5, Found: 1438.8 (M⁺+1).

5. Dpr²⁶ (β-DTPA)-Tyr²⁷, Nle^{28,31}-CCK(26-33): Dpr(β-DTPA)-Tyr-Nle-Gly-Trp-Nle-Asp-Phe-NH₂. (Compound 23). Molecular Weight, Calculated: 1372.6, Found: 1373.7 (M⁺+1).

6. DTPA-Tyr²⁷ Thr²⁸, Nle³¹-CCK(26-33): DTPA-Asp-Tyr-Thr-Gly-Trp-Nle-Asp-Phe-NH₂ (Compound 24). Molecular Weight, Calculated: 1389.5, Found: 1390.5 (M⁺+1).

### EXAMPLE 3

### Preparation of ¹¹⁵In Labelled Compounds 25 and 26

Peptides are dissolved in 5 mM NaHCO, at a concentration of 2.0 mg/ml. Labelling conditions are performed using a 1.5:1.0 molar ratio of ¹¹⁵In³⁺ (as InCl to peptide.

Labelling Procedure:

To 50 μl of peptide solution (100 μg peptide, 71.4 nmol) is added 23.7 μl (107.1 nmol)of a ¹¹⁵InCl₃ in 0.05N HCl (1.0 mg/ml) solution. Water is added to bring the final volume of the reaction to 200 μl. After 15 minutes at room temperature the solution is frozen and subsequently lyophilized to dryness. Dried ¹¹⁵In complexed peptide is re-dissolved in 10 mM NaHCO₃ and analyzed by reversed phase HPLC and by Mass Spectroscopy.

With the above mentioned labelling procedure compounds 25 (¹¹⁵In-DTPA-Asp-Tyr-Nle-Gly-Trp-Nle-Asp-Phe-NH₂) and 26 (¹¹⁵In-DTPA-DAsp-Tyr-Nle-Gly-Trp-Nle-Asp-Phe-NH₂) were prepared. HPLC analysis indicated that the peptides were >99′ complexed with ¹¹⁵In. Mass spectroscopy analysis yielded the expected molecular weight.

### EXAMPLE 4

### Receptor Affinity Studies with Unlabelled Compounds

Compounds 15, 16 and 18 are only used by comparison and are not in the scope of the present invention. Receptor autoradiography is performed on 10- and 20-μm thick cryostat sections of the various tumour samples, as described by Reubi et al. (Cancer Res. 1990, 50, 5969-5977).

Unlabelled CCK-8 (=Asp-Tyr(SO₃H)-Met-Gly-Trp-Met-Aso-Phe-NH₂, compound 16) and un-labelled desulfated CCK-8 (=Asp-Tyr-Met-Gly-Trp-Met-Asp-Phe-NH₂, compound 17) are obtained from Bachem AG, Bubendorf, Switzerland. Unlabelled CCK-10 analog (=D-Tyr-Gly-Asp-Tyr(SO₃H)-Nle-Gly-Trp-Nle-Asp-Phe-NH₂, compound 18) is obtained from Research Plus Laboratories, Bayonne, N.J., USA.

¹²⁵I-labelled peptides are prepared via the chloramine T iodination procedure, according to procedures as reported earlier by Greenwood et al. (Biochemical Journal 1963, 89, 114-123).

The [¹²⁵I-D-Tyr²⁴], Nle^{28,31}-CCK 24-33 labelled peptide 15 (=[¹²⁵I-D-Tyr]-Gly-Asp-Tyr(SO₃H)-Nle-Gly-Trp-Nle-Asp-Phe-NH₂) is separated by HPLC, using a reverse phase RC₁₈ column and butane-sulphonic acid as the eluent. The mono-¹²⁵iodinated compound is eluted as single peak from the HPLC and analysed by mass-spectrometry. Specific activity: 2000 Ci/mmol. The tissues are cut on a cryostat, mounted on microscopes slides, and then stored at −20° C. for at least 3 days to improve adhesion of the tissue to the slide. The slide-mounted tissue sections are allowed to reach room temperature and are preincubated in 50 mmol/l Tris-HCl, 130 mmol/l NaCl, 4.7 mmol/l KCl, 5 mmol/l MgCl₂, 1 mmol/l ethylene glycol-bi(β-aminoethylether)-N,N,N′,N′-tetraacetic acid, and 0.5% bovine serum albumin, pH 7.4 (preincubation solution), for 30 min. at 25° C. The slides are then incubated in a solution containing the same medium as the preincubation solution except the bovine serum albumin is omitted, and the following compounds are added: 20000 dpm/100 μl of ¹²⁵I-CCK, 0.025% bacitracin, 1 mmol/l dithiothreitol, 2 μg/ml chymostatin, and 4 μg/ml leupeptin, pH=6.5. The slides are incubated at room temperature with the radioligand for 150 min., as described by Mantyh et al. (Gasteroenterology 1994, 107, 1019-30). To estimate non-specific binding, paired serial sections are incubated as described above, except that CCK-8 (sulfated) is added to the incubation medium. After the incubation, the slides are rinsed with four washes of 30 sec each in ice-cold preincubation solution, pH 7.4, dipped in ice-cold water, and then quickly dried in a refrigerator under a stream of cold air. The sections are subsequently exposed to a 3H-Ultrofilm for 1 week, to detect the precise location of the radioactivity.

In all tumours, displacement experiments using successive sections of a tumour are performed with increasing concentrations of various biologically active or inactive peptides (see the above-mentioned publication by Reubi et al.). In comparison with sulfated CCK, unsulfated CCK, as well as somatostatin are used.

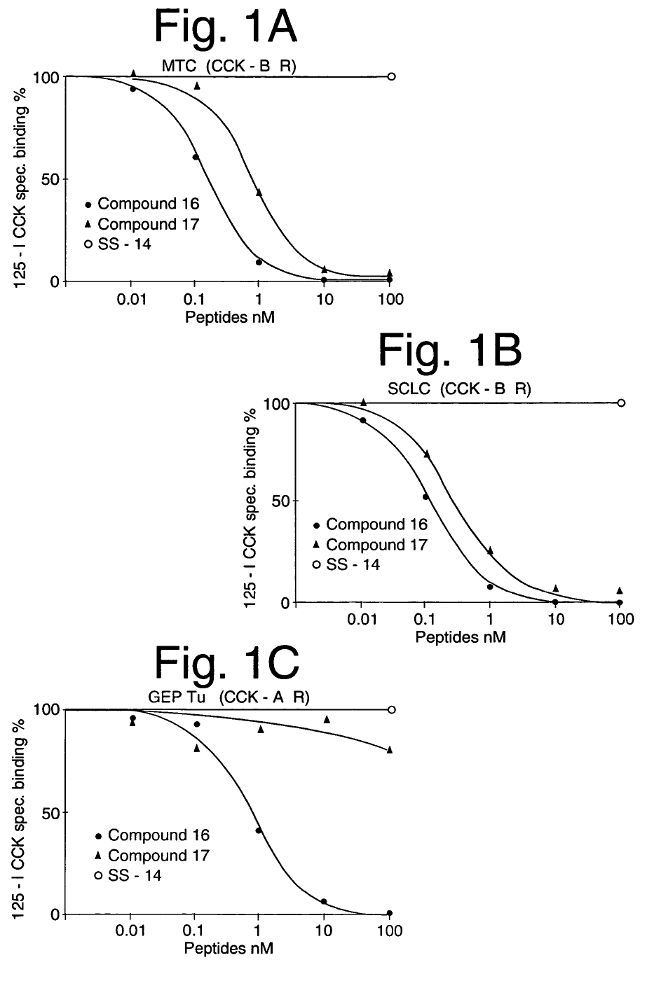

The FIG. 1 attached shows displacement curves of [¹²⁵I]-CCK-10 analog (compound 15) binding to tissue sections from three different tumours: A=medullary thyroid carcinoma (MTC) and B=small cell lung carcinoma (SCLC) and C=Gastro entero pancreatic tumour (GEP-Tu).

Tissue sections are incubated with 20,000 cpm/100 μl [¹²⁵I]-CCK-10 and increasing concentrations of unlabelled CCK-8 (unsulfated)(▴), CCK-8 (sulfated) (•) or somastotatin (∘). Each point represents the optical density of binding measured in the tumour area. Non-specific binding is subtracted from all values. In all cases, complete displacement of the ligand is achieved by sulfated CCK and unsulfated CCK is inactive in GEP-TU, whereas somastotatin is inactive in the nanomolar range for all three types of tumours.

This experiment shows that MTC and SCLC, expressing CCK-B tumours and that GEP-TU, expressing CCK-A receptors, can respectively be detected with both sulfated and unsulfated radiolabelled CCK or with sulfated radiolabelled CCK only. Therefore it can be concluded that tumours expressing CCK-B receptors can selectively be detected with unsulfated radiolabelled CCK without disturbing effects of CCK-A receptor expressing tissues or tumours.

### EXAMPLE 5

### Receptor Affinity Studies with Unlabelled DTPA Substituted Compounds 19-24

The experiments are performed as described in Example 4. The displacement curves of DTPA substituted CCK-analogs, prepared as described in Example 2, are measured in order to determine the effect of the DTPA group and of substitution of some amino acids by other amino acids.

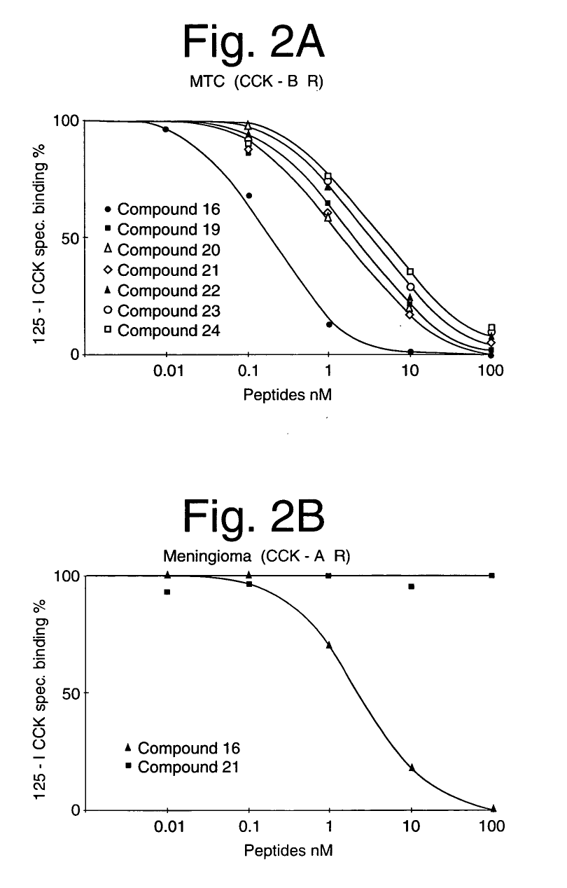

FIG. 2 attached shows displacement curves of [¹²⁵I]-CCK-10 (compound 15) binding to tissues from two different tumours: A=medullary thyroid carcinoma (MTC) and B=Meningioma of different DTPA-substituted unsulfated CCK-8. Tissue sections are incubated with 20,000 cpm/100 μl ¹²⁵ I]-CCK-10 and increasing concentrations of compound 16 (CCK-8 (sulfated))(•), compound 19 (▪), compound 20 (Δ), compound 21 (⋄), compound 22 (▴), compound 23 (□) and compound 24(∘).

Each point represents the optical density of binding measured in the tumour area. Non-specific binding is subtracted from all values. In MTC having CCK-B receptors complete displacement of the ligand is achieved by all compounds. In Meningioma, having CCK-A receptors displacement is only achieved by the sulfated compound 16.

This experiment shows that the CCK analogs of the invention retain affinity towards the CCK-B receptor after substitution with DTPA and after substitution of the amino acids in the 26, 28 and 31 position, and to not have affinity towards the CCK-A receptor.

### EXAMPLE 6

### Receptor Affinity Studies with ¹¹⁵ n-DTPA Substituted Compounds 25 and 26

The experiments are performed as described in Example 4. The displacement curves of ¹¹⁵In-DTPA substituted CCK-analogs, prepared as described in Example 3, are measured in order to determine the effect of the ¹¹⁵In-DTPA group and of substitution of some amino acids by other amino acids.

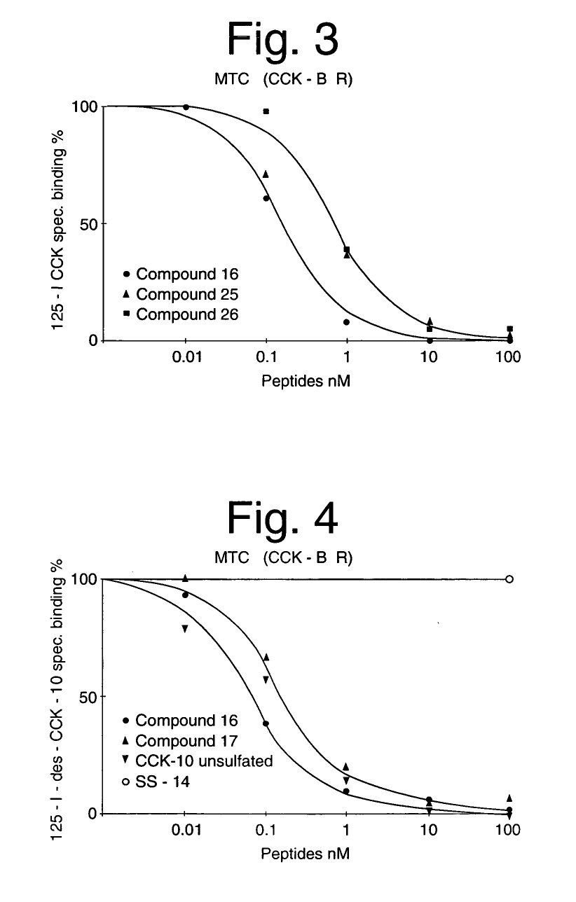

FIG. 3 attached shows displacement curves of [¹²⁵I]-CCK-10 (compound 15) binding to tissues from medullary thyroid carcinoma (MTC) of two different ¹¹⁵In-DTPA substituted desulfated CCK-8 analogs. Tissue sections are incubated with 20,000 cpm/100 μl [¹²⁵I]-CCK-10 and increasing concentrations of compound 16 (CCK-8 (sulfated))(•), compound 25 (▴) and compound 26 (▪). Each point represents the optical density of binding measured in the tumour area. Non-specific binding is subtracted from all values. In all cases, complete displacement of the ligand is achieved.

This experiment shows that the CCK analogs of the invention retain affinity towards the receptor after substitution with ¹¹⁵In-DTPA and after substitution of the amino acids in the 26, 28 and 31 position.

### EXAMPLE 7

The experiments are performed as described in Example 4. Instead of the [¹²⁵I]-CCK-10 analog the desulfated [¹²⁵]-CCK-10 compound (=¹²⁵I[D-Tyr-Gly-Asp-Tyr-Nle-Gly-Trp-Nle-Asp-Phe-NH₂]) is prepared by iodination of compound 12 as described in Example 1. The two mono-iodinated compounds 13 and 14 obtained after iodination are separated by HPLC, using a reverse phase RC₁₈ column and butane-sulphonic acid as the eluent. The two mono-¹²⁵-iodinated compounds are eluted as a single peak from the HPLC and are analysed by mass-spectrometry.

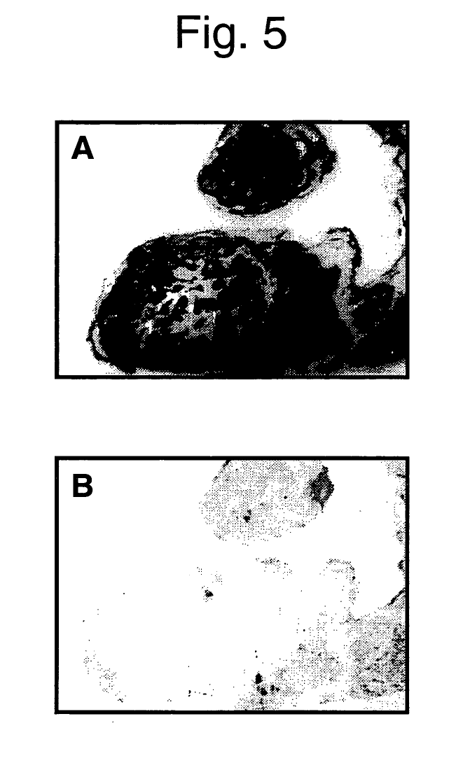

The FIG. 4 attached shows displacement curves of [¹²⁵I]-desulfated-CCK-10 binding (compound 13 or 14) to tissue sections from medullary thyroid carcinoma (MTC). Tissue sections are incubated with 20,000 cpm/100 μl [¹²⁵I]-desulfated-CCK-10 and increasing concentrations of compound 17 (CCK-8 (unsulfated)) (▴), compound 16 (CCK-8 (sulfated)) (•), CCK-10 (unsulfated) (▾) or somastotatin (∘). Each point represents the optical density of binding measured in the tumour area. Non-specific binding is subtracted from all values. In all cases, complete displacement of the ligand is achieved by sulfated and unsulfated CCK, whereas somastotatin is inactive in the nanomolar range. The two different mono ¹²⁵I-iodinated compounds appear to have the same affinity. FIG. 5 attached shows the autoradiogram of the binding of ¹²⁵I desulfated CCK-10 ligand to CCK-B receptors in MTC. A=Autoradiogram showing total binding of the ligand; B=Autoradiogram showing non-specific binding (in the presence of 10⁻⁶ desulfated CCK-10 analog).

1. A method of detecting and localizing malignant tumours and their metastases in tissues, which in healthy condition do not contain disturbing quantities of CCK-receptors, in the body of a human being which comprises (i) administering to said being a composition comprising, in a quantity sufficient for external imaging, a peptide of the general formula
$$H - {( Xaa )}_{n} - {( Xbb )}_{m} - Tyr - Xcc - {Gly}_{5} - Trp - Xdd - Asp - Phe - {R}_{2} \qquad ( I )$$
or an acid amide thereof, formed between a free NH₂-group of an amino acid moiety and R₁COOH, wherein
R₁ is a (C₁-C₃)alkanoyl group, an arylcarbonyl group, or an aryl-(C₁-C₃) alkanoyl group;
or a lactam thereof, formed between a free NH₂ group of an amino acid moiety and a free CO₂H group of another amino acid moiety;
or a conjugate thereof with avidin or biotin;
wherein:
(Xaa)ₙ stands for 0 to 25 amino acid moieties which are equal or different and are selected from Ala, Leu, Asn, Dpr, Gln, Glu, Ser, Ile, Met, His, Asp, Lys, Gly, Thr, Pro, Pyr, Arg, Tyr, Trp, Val and Phe;
m=0 or 1;
Xbb is Asp, Dpr, Glu or Pyr; with the proviso that Xbb can only be Pyr when n=0;
Xcc is Met, Leu or Nle;
Xdd is Met, Leu or Nle; and
R₂ is a hydroxy group, an acetoxy group or an amino group;
said peptide being labelled with (a) a radioactive metal isotope selected from the group consisting of ^{99m}Tc, ²⁰³ Pb, ⁶⁷Ga, ⁶⁹Ga, ⁷²As, ¹¹¹In, ^{113m}In, ⁹⁷Ru, ⁶²Cu, ⁶⁴Cu, ⁵²Fe, ^{52m}Mn and ⁵¹Cr, or (b) with a paramagnetic metal atom selected from the group consisting of Cr, Mn, Fe, Co, Ni, Cu, Pr, Nd, Sm, Yb, Gd, Tb, Dy, Ho and Er, or (c) with a radioactive halogen isotope, selected from ¹²³I, ¹³¹Br, ⁷⁵Br, ⁷⁶Br, ⁷⁷Br and ⁸²Br, and thereupon (ii) subjecting said being to external imaging, by radioactive scanning or by magnetic resonance imaging, to determine the targeted sites in the body of said being in relation to the background activity, in order to allow detection and localization of said tumours in the body.

2. A method of intraoperatively detecting and localizing malignant tumours in tissues, which in healthy condition do not contain disturbing quantities of CCK-receptors, in the body of a human being, which comprises (i) administering to said being a composition comprising, in a quantity sufficient for detection by a gamma detecting probe, a peptide of the general formula I as defined in claim 1 or an acid amide thereof, formed between a free NH₂-group of an amino acid moiety and R₁COOH;
or a lactam thereof, formed between a free NH₂ group of an amino acid moiety and a free CO₂H group of another amino acid moiety;
or a conjugate thereof with avidin or biotin; wherein (Xaa)ₙ, Xbb, Xcc, Xdd, m, R₁ and R₂ have the same meanings as in claim 1, said peptide being labelled with ¹⁶¹Tb, ¹²³I or ¹²⁵I and thereupon (ii), after allowing the active substance to be bound and taken up in said tumours and after blood clearance of radioactivity, subjecting said being to a radioimmunodetection technique in the relevant area of the body of said being, by using a gamma detecting probe.

3. A method for the therapeutic treatment of malignant tumours in tissues, which in healthy condition do not contain substantial quantities of CCK-receptors, in the body of a human being, which comprises administering to said being a composition comprising, in a quantity effective for combating or controlling tumours, a peptide of the general formula I as defined in claim 1 or an acid amide thereof, formed between a free NH₂-group of an amino acid moiety and R₁COOH;
or a lactam thereof, formed between a free NH₂ group of an amino acid moiety and a free CO₂H group of another amino acid moiety;
or a conjugate thereof with avidin or biotin; wherein (Xaa)ₙ, Xbb, Xcc, Xdd, m, R₁ and R₂ have the same meanings as in claim 1, said peptide being labelled with an isotope selected from the group consisting of ¹⁸⁶Re, ¹⁸⁸Re, ⁷⁷As ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ⁵¹Tb, ¹⁰⁹Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹ Yb, ¹⁷⁵Yb, ¹⁷⁷Lu, ¹⁰⁵Rh, ¹¹¹Ag, ¹²⁴I and ¹³¹I.

4. A method as claimed in claims 1, 2, or 3, characterized in that the tumours and the metastasis thereof to be detected, localized or therapeutically treated are selected from the group consisting of Small Cell Lung Carcinoma, Medullary Thyroid Carcinoma, Breast Carcinoma, Stromal Ovarian Carcinoma Muscle Sarcoma.

5. A method as claimed in claim 1 characterized in that the tumours and the metastasis thereof to be detected, localized or therapeutically treated are selected from the group consisting of Small Cell Lung Carcinoma and Medullary Thyroid Carcinoma.

6. A method as claimed in claims 1, 2, or 3 characterized in that said peptide is selected from the group consisting of H-DTyr-Gly-Asp-Tyr-Nle-Gly-Trp-Nle-Asp-Phe-NH₂, H-Asp-Tyr-Met-Gly-Trp-Met-Asp-Phe-NH₂, H-Asp-Tyr-Nle-Gly-Trp-Nle-Asp-Phe-NH₂, H-DAsp-Tyr-Nle-Gly-Trp-Nle-Asp-Phe-NH₂, H-DAsp-Tyr-Met-Gly-Trp-Met-Asp-Phe-NH₂ and H-Dpr-Tyr-Nle-Gly-Trp-Nle-Asp-Phe-NH₂, and preferably is H-Asp-Tyr-Nle-Gly-Trp-Nle-Asp-Phe-NH₂ or H-DAsp-Tyr-Nle-Gly-Trp-Nle-Asp-Phe-NH₂.

7. A method as claimed in claims 1, 2, or 3 which comprises administering to said living being a composition comprising a labelled peptide as defined in said preceding claims, wherein said peptide is labelled with a radioactive halogen isotope selected from the group consisting of ¹²³I, ¹²⁴I, ¹²⁵I, ¹³¹I, ⁷⁵I, ⁷⁶Br, ⁷⁷Br and ⁸²Br, said radioactive halogen isotope being attached to a Tyr or Trp moiety of the peptide, or to the aryl group of substituent R₁.

8. A method as claimed in any of the preceding claims 1-3 which comprises administering to said living being a composition comprising a labelled peptide as defined in said preceding claims, wherein said peptide is labelled with a metal atom selected from (a) the group consisting of the radioactive isotopes ^{99m}Tc, ²⁰³Pb, ⁶⁶Ga, ⁶⁷Ga, ⁶⁸Ga, ⁷²As, ¹¹¹In, ^{113m}In, ^{114m}In, ⁹⁷Ru, ⁶²Cu, ⁶⁴Cu, ⁵²Fe, ^{52m}n, ⁵¹Cr, ¹⁸⁶Re, ¹⁸⁸Re, ⁷⁷As, ⁹⁰Y, ⁶⁷Cu, ¹⁶⁹Er, ^{117m}Sn, ¹²¹Sn, ¹²⁷Te, ¹⁴²Pr, ¹⁴³Pr, ¹⁹⁸Au, ¹⁹⁹ Au, ¹⁴⁹Tb, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁶⁶Ho, ¹⁷²Tm, ¹⁶⁹Yb, ¹⁷⁵Yb, ¹⁷⁷Lu, ¹⁰⁵Rh and ¹¹¹Ag or (b) the group consisting of the paramagnetic metal atoms Cr, Mn, Fe, Co, Ni, Cu, Pr, Nd, Sm, Yb, Gd, Tb, Dy, Ho and Er; said metal atom being attached to the peptide by means of a chelating group chelating said atom, which chelating group is bound by an amide bond or through a spacing group to the peptide molecule.

9. A method as claimed in claim 8, wherein said composition comprises a peptide labelled with a metal atom, chelated by an NₜS₍₄₋ₜ₎ tetradentate chelating agent, wherein t=2-4, or by a chelating group derived from ethylene diamine tetra-acetic acid (EDTA), diethylene triamine penta-acetic acid (DTPA), cyclohexyl 1,2-diamine tetra-acetic acid (CDTA), ethyleneglycol-0,0′-bis(2-aminoethyl)-N,N,N′,N′-tetra-acetic acid (EGTA), N,N-bis(hydroxybenzyl)-ethylenediamine-N,N-diacetic acid (HBED), triethylene tetramine hexa-acetic acid (TTHA), 1,4,7,10-tetraazacyclododecane-N,N′,N″,N′″-tetra-acetic acid (DOTA), hydroxyethyldiamine triacetic acid (HEDTA), 1,4,8,11-tetra-azacyclo-tetradecane-N,N′,N″,N′″-tetra-acetic acid (TETA), substituted DTPA, substituted EDTA, or from a compound of the general formula 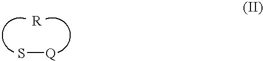
wherein R is a branched or non-branched, optionally substituted hydrocarbyl radical, which may be interrupted by one or more hetero-atoms selected from N, O and S and/or by one or more NH groups, and
Q is a group which is capable of reacting with an amino group of the peptide and which is preferably selected from the group consisting of carbonyl, carbimidoyl, N-(C₁-C₆) alkylcarbimidoyl, N-hydroxycarbimidoyl and N-(C₁-C₆)alkoxycarbimidoyl; and
wherein said optionally present spacing group is a biotinyl moiety or has the general formula 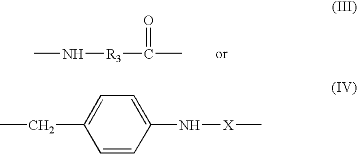
wherein R₃ is a C₁-C₁₀ alkylene group, a C₁-C₁₀ alkylidene group or a C₂-C₁₀ alkenylene group, and x is a thiocarbonyl group or a group of the general formula 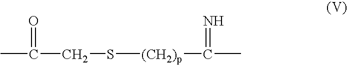
wherein p is 1-5.

10. The use of a labelled peptide as defined in any of the preceding claims 1 or 2 for preparing a diagnostic composition for detecting and localizing malignant human tumours, including the metastases thereof, in the body of a human being.

11. The use of a labelled peptide as defined in any of the preceding claims 3-5 for preparing a pharmaceutical composition for the therapeutic treatment of malignant human tumours, including the metastases thereof, in the body of a human being.

12. A pharmaceutical composition to be used for the method as claimed in claim 1, 2 or 3, comprising in addition to a pharmaceutically acceptable carrier material and, if desired, at least one pharmaceutically acceptable adjuvant, as the active substance, in a quantity sufficient for external imaging, respectively detection by a gamma detecting probe or for combating or controlling tumours, a peptide of the general formula I as defined in claim 1 or an acid amide thereof, formed between a free NH₂-group of an amino acid moiety and R₁COOH;
or a lactam thereof, formed between a free NH₂ group of an amino acid moiety and a free CO₂H group of another amino acid moiety;
or a conjugate thereof with avidin or biotin; wherein (Xaa)ₙ, Xbb, Xcc, Xdd, m, R₁ and R₂ have the same meanings as in claim 1, said peptide being labelled with (a) a radioactive metal isotope selected from the group consisting of ^{99m}Tc, ²⁰³Pb, ⁶⁶Ga, ⁶⁷Ga, ⁶⁸Ga, ⁷² As, ¹¹¹In, ^{113m}In, ^{114m}In, ⁹⁷Ru, ⁶²Cu, ⁶⁴Cu, ⁵²Fe, ^{52m}Mn, ⁵¹Cr, ¹⁸⁶Re, ¹⁸⁸Re, ⁷⁷As, ⁹⁰Y, ⁶⁷Cu, ¹⁶⁹Er, ^{117m}Sn, ¹²¹Sn, ¹²⁷Te, ¹⁴²Pr, ¹⁴³Pr, ¹⁹⁸Au, ¹⁹⁹A, ¹⁴⁹Tb, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁶⁶Ho, ¹⁷²Tm, ¹⁶⁹Yb, ¹⁷⁵Yb, ¹⁷⁷Lu, ¹⁰⁵Rh and ¹¹¹Ag, or (b) with a paramagnetic metal atom selected from the group consisting of Cr, Mn, Fe, Co, Ni, Cu, Pr, Nd, Sm, Yb, Gd, Tb, Dy, Ho and Er, or (c) with a radioactive halogen isotope, selected from ¹²³I, ¹³¹I, ⁷⁵Br, ⁷⁶Br, ⁷⁷Br and ⁸²Br.

13. A composition as claimed in claim 12, characterized in that the active substance is a derivatized peptide selected from the group consisting of DTPA-Asp-Tyr-Met-Gly-Trp-Met-Asp-Phe-NH₂, DTPA-Asp-Tyr-Nle-Gly-Trp-Nle-Asp-Phe-NH₂, DTPA-DAsp-Tyr-Nle-Gly-Trp-Nle-Asp-Phe-NH₂, DTPA-DAsp-Tyr-Met-Gly-Trp-Met-Asp-Phe-NH₂ and Dpr(β-DTPA)-Tyr-Nle-Gly-Trp-Nle-Asp-Phe-NH₂, said derivatized peptide being labelled with a metal atom as defined in claim 8.

14. A composition as claimed in claim 13, characterized in that said derivatized peptide is DTPA-Asp-Tyr-Nle-Gly-Trp-Nle-Asp-Phe-NH₂ or DTPA-DAsp-Tyr-Nle-Gly-Trp-Nle-Asp-Phe-NH₂.

15. A pharmaceutical composition to be used for the method as claimed in claim 2, comprising in addition to a pharmaceutically acceptable carrier material and, if desired, at least one pharmaceutically acceptable adjuvant, as the active substance, in a quantity sufficient for intraoperatively detecting and localizing malignant tumours, a peptide selected from the group consisting of [¹²⁵I-D-Tyr]-Gly-Asp-Tyr-Nle-Gly-Trp-Nle-Asp-Phe-NH₂ and D-Tyr-Gly-Asp-[¹²⁵I-Tyr]-Nle-Gly-Trp-Nle-Asp-Phe-NH_{2.}

16. A labelled peptide to be used as an active ingredient in a composition as claimed in claim 12, 13 and 14, said peptide being labelled with a metal atom as defined in claim 8.

17. A labelled peptide to be used as an active ingredient in a composition as claimed in claim 15, said peptide having the chemical structure as defined in claim 15.

18. A method of preparing a metal atom-labelled peptide as claimed in claim 16, characterized in that a derivatized peptide, comprising a peptide of the general formula I as defined in claim 1 or an acid amide thereof, formed between a free NH₂-group of an amino acid moiety and R₁COOH;
or a lactam thereof, formed between a free NH₂ group of an amino acid moiety and a free CO₂H group of another amino acid moiety;
or a conjugate thereof with avidin or biotin; wherein (Xaa)ₙ, Xbb, Xcc, Xdd, m, R₁ and R₂ have the same meanings as in claim 1, derivatized with a chelating group bound by an amide bond or through a spacing group to the peptide molecule, is reacted with a metal atom as defined in claim 8 in the form of a salt or of a chelate, bound to a comparatively weak chelator, in order to form a complex.

19. A derivatized peptide as defined in claim 18, comprising a peptide of the general formula I as defined in claim 1 or an acid amide thereof, formed between a free NH₂-group of an amino acid moiety and R₁COOH;
or a lactam thereof, formed between a free NH₂ group of an amino acid moiety and a free CO₂H group of another amino acid moiety;
or a conjugate thereof with avidin or biotin; wherein (Xaa)ₙ, Xbb, Xcc, Xdd, m, R₁ and R₂ have the same meanings as in claim 1, derivatized with a chelating group bound by an amide bond or through a spacing group to the peptide molecule.

20. A derivatized peptide as defined in claim 19, selected from the group consisting of DTPA-Asp-Tyr-Met-Gly-Trp-Met-Asp-Phe-NH₂, DTPA-Asp-Tyr-Nle-Gly-Trp-Nle-Asp-Phe-NH₂, DTPA-DAsp-Tyr-Nle-Gly-Trp-Nle-Asp-Phe-NH., DTPA-DAsp-Tyr-Met-Gly-Trp-Met-Asp-Phe-NH₂ and Dpr(β-DTPA)-Tyr-Nle-Gly-Trp-Nle-Asp-Phe-NH₂, said derivatized peptide preferably being DTPA-Asp-Tyr-Nle-Gly-Trp-Nle-Asp-Phe-NH₂ or DTPA-DAsp-Tyr-Nle-Gly-Trp-Nle-Asp-Phe-NH_{2.}

21. A kit for preparing a radiopharmaceutical composition, comprising (i) a derivatized peptide as claimed in claim 19 and 20, to which derivatized peptide, if desired, an inert pharmaceutically acceptable carrier and/or formulating agents and/or adjuvants is/are added, (ii) a solution of a salt or chelate of a metal selected from the group consisting of the radioactive isotopes ²⁰³Pb, ⁶⁶Ga, ⁶⁷Ga, ⁶⁸Ca, ⁷²As, ¹¹¹In, ^{113m}In, ^{114m}In, ⁹⁷Ru, ⁶²Cu, ^{99m}Tc, ¹⁸⁶Re, ¹⁸⁸Re, ⁶⁴Cu, ⁵²Fe, ^{52m}Mn, ⁵¹Cr, ⁷⁷As, ⁹⁰Y, ⁶⁷Cu, ¹⁶⁹Er, ^{117m}Sn, ¹²¹Sn, ¹²⁷Te, ¹⁴²Pr, ¹⁴³Pr, ¹⁹⁸Au, ¹⁹⁹Au, ¹⁴⁹Tb, ¹⁶¹Tb, ¹⁰⁹Pd, ¹⁶⁵Dy, ¹⁴⁹Pm, ¹⁴⁹Pm, ¹⁵³Sm, ¹⁵⁷Gd, ¹⁵⁶Ho, ¹⁷²Tm, ¹⁶⁹Yb, ¹⁷⁵Yb, ¹⁷⁷Lu, ¹⁰⁵Rh and ¹¹¹Ag, and (iii) instructions for use with a prescription for reacting the ingredients present in the kit.

22. A kit for preparing a radiopharmaceutical composition, comprising (i) a derivatized peptide as claimed in claim 19 and 20, to which derivatized peptide, if desired, an inert pharmaceutically acceptable carrier and/or formulating agents and/or adjuvants is/are added, (ii) a reducing agent, and, if desired, a chelator, said ingredients (i) and (ii) optionally being combined, and (iii) instructions for use with a prescription for reacting the ingredients of the kit with ^{99m}Tc in the form of a pertechnetate solution or with ¹⁸⁶Re or ¹⁸⁸Re in the form of a perrhenate solution.
